(19) United States
(12) Patent Application Publication
(10) Pub. No.: US 2007/0042005 A1
(43) Pub. Date: Feb. 22, 2007

(54) Pharmaceutical composition comprising fungal cell or fragment thereof

(76) Inventors: Michael Crothers, Manchester (GB); Stephen C. Duckham, Merseyside (GB); Gordon Nelson, Cheshire (GB)

Correspondence Address: BOYLE FREDRICKSON NEWHOLM STEIN & GRATZ, S.C., 250 E. WISCONSIN AVENUE, SUITE 1030, MILWAUKEE, WI, 53202, US

(21) Appl. No.: 10/550,837

(22) PCT Filed: Mar. 12, 2004
(86) PCT No.: PCT/GB04/01072

§ 371 Date: Aug. 2, 2006

(30) Foreign Application Priority Data
Mar. 26, 2003 (GB) 0306933.3

(51) Int. Cl. A61K 39/00; A61K 36/06

(52) U.S. Cl. 424/274.1; 424/195.16

(57) ABSTRACT

The present invention relates to compositions, methods and uses of such compositions, the compositions comprising a pharmaceutically active compound and a non-encapsulating adjuvant, wherein the adjuvant comprises a fungal cell or a fragment thereof.

The present invention relates to pharmaceutical compositions, methods comprising the use of such compositions and packaged compositions.

The epithelium is a primary animal tissue and covers the body's internal and external surfaces, such as the mouth, oesophagus, and gastro-intestinal tract (GI tract). It functions as a semi-permeable barrier to separate the body from the external environment as well as to maintain distinct compartments within the body.

Organs and tissues depend on the assembly of polarised epithelial cells, arranged in sheets, for their physiological functions. Individual epithelial cells interact with each other as well as with the underlying extracellular matrix via membrane-associated proteins, eg. transmembrane proteins and cytoplasmic plaque proteins, that serve as connections to the cytoskeleton and junctional complexes.

Molecules cross the epithelium via two main pathways, namely the transcellular pathway and the paracellular pathway. The transcellular pathway includes endocytosis, passive diffusion, carrier-mediated transport and facilitated diffusion. The paracellular pathway comprises passive diffusion.

Owing to the nature of the cell membrane of epithelial cells, molecules which are not recognised by a carrier, such as hydrophilic compounds, cannot readily traverse the cell membrane and instead traverse the epithelium barrier via the paracellular pathway. Examples of such molecules include hydrophilic and charged drugs, peptides, amino acids etc. However, the transport of such molecules via this pathway is restricted by the presence of tight junctions (1).

Tight junctions serve as selective barriers and appear to be modulated by cellular processes which regulate the movement of molecules, such as hydrophilic molecules, across the epithelium, although the precise mode of operation of tight junctions has yet to be determined. In this regard, glucose has been hypothesised to cross the epithelium in part via the paracellular pathway (2). The epithelial barrier thus serves to keep potentially harmful agents out of the body and allow beneficial agents, such as ions, nutrients and water into the body.

The tight junction allows the passage of small hydrophilic compounds but acts as a barrier to larger hydrophilic ones. It forms an intramembrane diffusion barrier that restricts the intermixing of apical and basolateral membrane components (3,4). Its function is complex which is reflected in its multiprotein architecture. The tight junction is composed of a group of transmembrane and cytosolic proteins that interact not only with each other but also with the cell membrane and the cytoskeleton (5).

Many useful pharmaceutically active compounds are hydrophilic and cannot therefore be administered orally, which is the most convenient mode of drug delivery. For example, cefoxitin, a hydrophilic antibiotic, has an oral bioavailability of <5% in animals owing to poor intestinal permeability (6) and is currently only marketed as an intravenous formulation. Manufacturers have therefore sought to improve the bioavailability of their drugs. Enalaprilat, an angiotensin—converting enzyme inhibitor, is also poorly absorbed orally and is marketed as an intravenous formulation. Enalaprilat was chemically modified to produce a more lipophilic pro-drug, enalapril, which is more readily absorbed orally across the internal epithelium (7) and converted to the active, enalaprilat, thereby achieving therapeutic concentrations of the active hydrophilic agent after oral administration. Other approaches used by manufacturers involve re-engineering the drug so that it is a substrate, or is at least linked to a substrate, for a carrier involved in carrier mediated transport across the epithelial cell membrane (8). However, once an active compound has been discovered, it is costly both in time, resources and financial investment to further improve the bioavailability of the compound by further modification thereto. Furthermore, the active agent may be subject to degradation via intracellular enzymes and/or other intracellular interactions once it has been carried across the cell membrane.

Consequently, it would be advantageous to improve the bioavailability of drugs via one or more of the main pathways. Manufacturers have therefore turned their attention to the paracellular pathway. More particularly, manufacturers have sought to identify compounds (paracellular permeability enhancers (PPEs)) that selectively open the tight junction for co-administration with for example hydrophilic pharmaceutically active compounds.

However, such PPEs are generally toxic in vivo and damage the integrity of the epithelial cell membranes.

Palmitoyl carnitine has been found to increase the bioavailability of cefoxitin in rats, however, the effect was associated with mucosal damage (9).

Hormones and neurotransmitters such as vasopressin, angiotensin II and epinephrine have also been found to increase paracellular permeability in hepatocytes whilst cytokines, such as tumor necrosis factor, have also been found to increase epithelial permeability by modulating the tight junctions. However, these factors have a response time that is too long to be considered as useful PPEs.

Borchard et al., (10) have studied the potency of polyacrylic acid derivative carbomer (Carbopol (RTM) 934P) and chitosan-glutamate with Caco-2 cell cultures for use in the delivery of drugs. Their results show that whilst the carbomer may be effective, chitosan-glutamate does not suffice to permit paracellular transport of a drug and damages the integrity of the cells (11).

According to the US Food and Drug Administration's (FDA's) Biopharmaceutics Classification System (BCS), drug products are classified into four groups based on the ability of a given drug substance to permeate biological membranes and its aqueous solubility: Class I drugs are highly permeable, highly soluble; Class II drugs are highly permeable, poorly soluble; Class III drugs are poorly permeable, highly soluble; and Class IV drugs are poorly permeable, poorly soluble (The Biopharmaceutics classification system (BCS) guidance, Center for Drug Evaluation and Research, US Food and Drug Administration (FDA), 2001, www.fda.gov/cder). A drug substance is considered “highly soluble” when the highest dose strength is soluble in 250 ml water over a pH range 1 to 7.5, and “highly permeable” when the extent of absorption in humans is determined to be 90% of an administered dose, based on mass balance or related to an intravenous reference dose. For a rapidly dissolving tablet, 85% of the labelled amount of drug substance must dissolve within 30 minutes. Thus, for rapidly dissolving solid oral dosage forms, the dose-to-solubility ratio (D:S) of the drug must be 250 ml over pH range of 1 to 7.5. Class I drug substances, which possess both high permeability through biological membranes and good solubility in water, have the preferred physicochemical properties. Most new chemical identities are water-insoluble lipophilic compounds or, in other words, Class II or Class IV compounds which are traditionally difficult to formulate into usable pharmaceutical products. (Cyclodextrin-based Drug Delivery, Loftsson, T., and O'Fee, R., 2002, Business Briefing: Pharmatech, p 136-140).

It would therefore be advantageous to improve the bioavailability of pharmaceutically active compounds. It is an object of the present invention to overcome or alleviate one or more of the problems associated with the prior art.

In accordance with a first aspect of the present invention there is provided a composition comprising a pharmaceutically active compound and a non-encapsulating adjuvant, wherein the adjuvant comprises a fungal cell or a fragment thereof.

The inventors have surprisingly discovered that the addition of a fungal cell or a fragment thereof as an adjuvant increases the permeability of tight junctions between the cells of a subject when administered, without compromising cell viability. The present invention thus provides means for increasing the bioavailability of pharmaceutically active compounds via the paracellular pathway by incorporating an adjuvant which increases the permeability of the tight junctions of the pathway.

The fragment of fungal cell may comprise a fungal cell wall, such as a ghost cell, or a part thereof.

The term “non-encapsulating” as used herein relates to an adjuvant wherein at least some of the pharmaceutically active compound is not encapsulated by the fungal cell or fungal cell fragment. Encapsulated compounds are described in WO 00/69440. However, further pharmaceutically active compounds of the same or different type may be present in the composition, including compounds encapsulated in the or a fungal cell or fungal cell fragment.

The term “pharmaceutically active compound” as used herein is meant to include any therapeutic or otherwise active agent, e.g. a pharmaceutical compound or chemical.

The pharmaceutically active compound may be hydrophilic, hydrophobic or may comprise hydrophilic and hydrophobic moieties. Preferably, the pharmaceutically active compound is hydrophilic or substantially hydrophilic. The term ‘substantially hydrophilic’ as used herein is meant to include those compounds having hydrophilic and hydrophobic moieties wherein the hydrophilic moiety is predominant. The pharmaceutically active compound is preferably water soluble.

The pharmaceutically active compound may be in the form of a pro-drug. Pro-drugs may be any covalently bonded carrier that releases a compound in vivo when such pro-drug is administered. Pro-drugs are typically prepared by modifying functional groups in a way such that the modification is cleaved, preferably in vivo, yielding the parent pharmaceutically active compound.

The pharmaceutically active compound may comprise a peptide. The composition may additionally comprise an enzyme inhibitor to mitigate the loss of efficacy of peptide drugs via proteinases. Pharmaceutically active peptide compounds are well publicised as having low absorption properties in the G.I. tract, ie. low bioavailabilty.

Illustrative categories and specific examples of pharmaceutically active compounds useful in conjunction with the present invention include: anti-viral agents, analgesics, anaesthetics, anti-arthritics, anti-depressants, anti-diabetic agents, anti-inflammatory agents, anti-Parkinsonism drugs, anti-pruritics, cardiovascular drugs, anti-hypertensives, ACE inhibitors, vaccines, hormones, immunosuppressives, muscle relaxants, parasympatholytics, parasympathomimietics, psychostimultants, anti-tuberculosis agents, anti-tussives, histamine HI-receptor antagonists, histamine H2-receptor antagonists, decongestants, alkaloids, mineral supplements, laxatives, vitamins, antacids; ion exchange resins anti-lipidic agents, anti-pyretics, non steroidic anti-inflammatory (NSAI) substances, NSAI oxicam derivatives and appetite suppressants. Additional useful active medicaments include coronary dilators, cerebral dilators, peripheral vasodilators, anti-infectives, psychotropics, anti-manics, stimulants, gastrointestinal sedatives and bandages, anti-diarrhoeal and anti-constipation preparations, anti-anginal drugs, vasodilators, anti-hypertensive drugs, vasoconstrictors and migraine treatments, antibiotics, tranquillisers, anti-psychotics, anti-tumour drugs, anti-coagulants, and anti-thrombotic drugs, hypnotics, sedatives, anti-emetics, anti-nauseants, anti-convulsants, neuromuscular drugs, hyper- and hypoglycaemic agents, thyroid and anti-thyroid preparations, diuretics, anti-spasmodics, uterine relaxants, nutritional additives, anti-obesity drugs, anabolic drugs, erythropoietic drugs, anti-asthmatics, anti-histaminic or anti-cholinergic or opiate derivatives, cough suppressants, oral mucolytics, anti-uricemic drugs and the like. Other examples of actives are well known to a person skilled in the art.

In another composition according to the present invention, the pharmaceutically active compound is active via the paracellular pathway.

Preferably, the adjuvant is present in an amount effective to increase the permeability of the paracellular pathway. More preferably the adjuvant is be present in an amount of at least 0.5% by weight of composition.

The fungal cell or a fragment thereof may be derived from one or more fungi from the group comprising Mastigomycotina, Zygomycotina, Ascomycotina, Basidiomycotina and Deuteromycotina. Preferably, the fungal cell or a fragment thereof is derived from one or more fungi from Ascomycotina.

More preferably, the fungal cell or a fragment thereof is derived from yeasts. More preferably still, the fungal cell or a fragment thereof is derived from one or more of the group comprising Candida albicans, Blastomyces dermatitidis, Coccidioides immitis, Paracoccidioides brasiliensis, Penicillium marneffei and Saccharomyces cerevisiae. Even more preferably still, the fungal cell or a fragment thereof may be derived from Saccharomyces cerevisiae, such as Bakers yeast and Williams yeast (obtainable from Williams Bioenergy, 1300 South 2^{nd} Street, Pekin, Ill., 61555-00, USA).

The fungal cell or fungal cell fragment may be derived from a yeast which is grown continually or grown in a batch. Yeast grown continually is usually used for the production of ethanol for fuel purposes and is adapted to a high alcohol environment. Such yeast are termed ethanol yeast or biofuel yeast of which Williams yeast is an example. Most preferably the fungal cell or a fragment thereof is derived from a biofuel yeast.

In one composition according to the invention, the fungal cell or fungal cell fragment contains chitin and/or chitosan. The fungal cell or fungal cell fragment may also contain α- and/or β-glucans.

The fungal cell or fungal cell fragment may contain chitin in an amount of at least about 1%, preferably at least about 2%, more preferably at least about 5%, for example from about 5% to 10% or more, or from about 15 to 25% or more even up to 50% or 70-80% by dry weight. The percentage is preferably by dry weight of the fungal cell. The fungal cell or fungal cell fragment may also or instead contain chitosan in similar amounts

In a further composition according to the present invention, the fungal cell or fungal cell fragment may comprise at least one budding scar. Budding scars are formed when yeast cells bud during yeast cell division. When the bud and parent yeast cell separate a budding scar is formed on the cell wall surface of the parent cell where the bud was formed. Such scars are rich in chitin and/or chitosan. In this case the fungal cell or fungal cell fragment may comprise a plurality of budding scars.

If desired the fungal cell or cell fragment may be pre-treated to convert chitin to chitosan by de-acetylisation. One such pre-treatment comprises one or more of the steps set out below:

The fungal cell or fungal cell fragment, such as for example yeast cells or fungal fibres, are washed and treated with a 2N boiling solution of sodium hydroxide for one hour to dissolve protein from the outer layers of the cell wall and expose the underlying chitin or chitin and chitosan. Further de-acetylisation of the chitin may be effected by 40% sodium hydroxide solution.

The fungal cell or fungal cell fragment may be repeatedly washed until neutral pH is obtained and then preferably bleached by treatment with a solution of hydrogen peroxide (80 ml/l 37% H₂ 0₂+40 g/l NaOH+40 g/l sodium silicate) which may take place for two hours at room temperature.

Preferably, the treated fungal cell or fungal cell fragment is substantially white in colour. By comparison, untreated yeast, for example, have a cream to brown colour whilst filamentous fungi can be one of a variety of different colours.

The bleaching step may not be necessary when a substantially white fungal cell or fungal cell fragment is produced without the bleaching step.

When the adjuvant comprises a fungal cell, the fungal cell may be alive or dead. The adjuvant may comprise a plurality of fungal cells or fragments thereof, and may comprise a plurality of different types of fungal cells or fragments thereof.

The target for delivery of the compositions of the present invention may be any cellular structure having a tight junction. Preferably the target is the tight junctions found in epithelium, more preferably a mucous membrane.

The mucous membrane may be the membrane lining of the oral cavity or buccal cavity, tongue, stomach, small intestine (duodenum or jejunum), large intestine (colon), rectum, vagina, cervix, nose, naso-pharynx, or pulmonary system (trachea, larynx, bronchi, and lungs). The mucous membrane may be the membrane lining of the digestive system of animals, such as humans and other mammals including domestic pets and livestock.

The mucous membrane may be the membrane lining the oesophagus or stomach, where the composition of the present invention can be for pharmaceutical use, nutriceutical applications, or as an OTC medicine. The composition of the present invention can be incorporated in a one- or two-part gelatine capsule or other similar material to aid swallowing and prevent premature release of the active in the mouth or on the surface of the tongue. For example, protein-pump inhibitors (such as Omeprazole) may be encapsulated and formulated within a gelatine capsule to treat stomach ulcers.

The mucous membrane may be the membrane lining the small/large intestine where the composition of the present invention may be for pharmaceutical use or as an OTC medicine. To deliver to the small intestine, the composition of the present invention may be formulated with an acid-stable enteric coating which will break down only in alkaline conditions e.g. Eudragit (Rohm and Haas), Aquacoat (FMC) and Kollicaot (BASF). There are many examples of enteric coatings, as summarized in U.S. Pat. No. 4,755,387. The use of such enteric coatings allows drugs such as Fluoxetine (Prozac) to target the small intestine. Garlic, (which contains the active ingredient alacin which is known to have beneficial effects on the cardiovascular system and can reduce cholesterol), may be encapsulated and formulated with an enteric coating, to target delivery to the small intestine, thereby eliminating the powerful odour and taste characteristics associated with other garlic preparations.

Accordingly, the composition of the present invention may be formulated as a dry or liquid (emulsion or suspension) syrup, a sachet, a chewable, a chewing gum, an orodispersible, a dispersible effervescent, a dispersible tablet, a compressed buccal tablet, a compressed sublingual tablet, a chewable tablet, a melt-in-the-mouth, a lozenge, a paste, a powder, a gel, a tablet, a compressed sweet, a boiled sweet, a cream, a suppository, a snuff, a spray, an aerosol, a pessary, or an ointment.

In accordance with a second aspect of the present invention there is provided a composition comprising a pharmaceutically active compound and a non-encapsulating adjuvant, wherein the adjuvant comprises a fungal cell or a fragment thereof for use as a medicament.

In accordance with a further aspect of the present invention there is provided a packaged composition comprising at least two discrete components, the first component comprising a pharmaceutically active composition and the second component comprising a fungal cell or a fragment thereof.

The packaged composition may comprise a blister pack or the like. In one embodiment, the packaged composition comprises a blister pack comprising a deformable plastics sheet having at least two recesses formed therein for receiving the discrete components and a means for closing the recesses, retaining the discrete components therein. The means for closing the recesses may be a foil. In order to remove the discrete components from the pack, pressure is applied to the outer surface of a selected recess, causing the recess to deform, ejection of the component rupturing the foil in the process.

In accordance with a further aspect of the present invention, there is provided a method of administering a pharmaceutically active compound via the paracellular pathway comprising the use of a composition as described hereinabove.

In accordance with a further aspect of the present invention, there is provided the use of a non-encapsulating fungal cell or fungal cell fragment to augment the bioavailability of a pharmaceutically active compound.

In accordance with a further aspect of the present invention, there is provided the use of a composition as described hereinabove for alleviating the symptoms of and/or curing an ailment or disease.

Specific embodiments of the present invention will now be described, by way of example only, and with reference to the accompanying figures, in which:

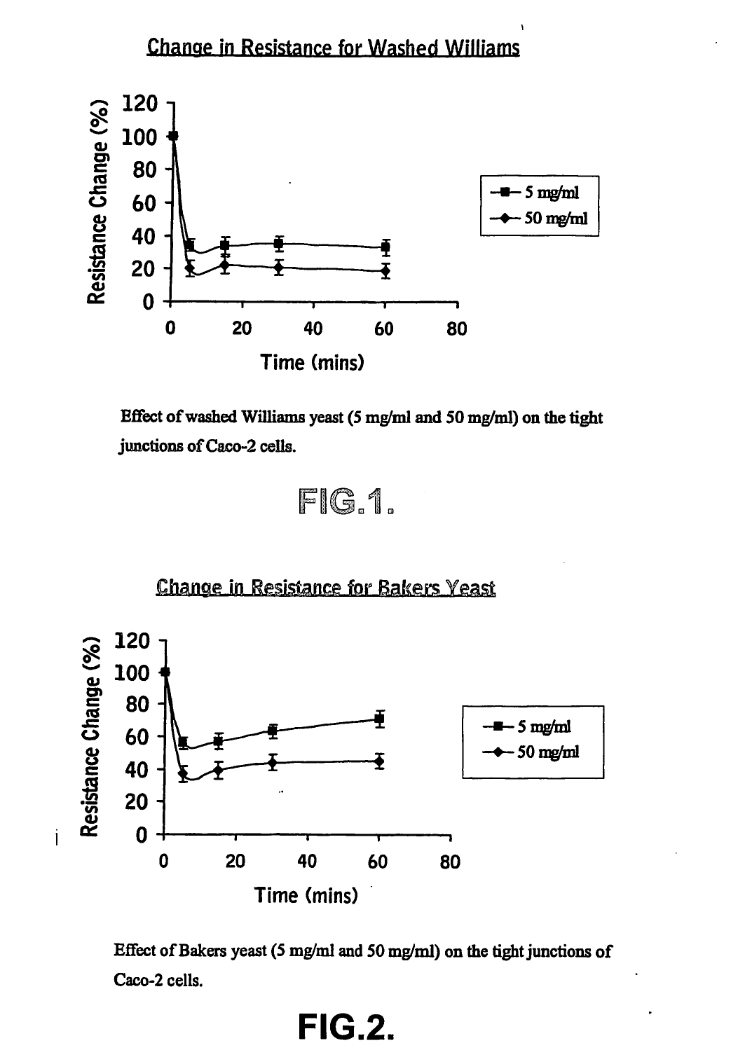

FIG. 1 shows the percentage change in resistance at selected time periods for Washed Williams (5 and 50 mg/ml) yeast on the tight junctions of Caco-2 cells;

FIG. 2 shows the percentage change in resistance at selected time periods for Balkers yeast (5 and 50 mg/ml) on the tight junctions of Caco-2 cells; and,

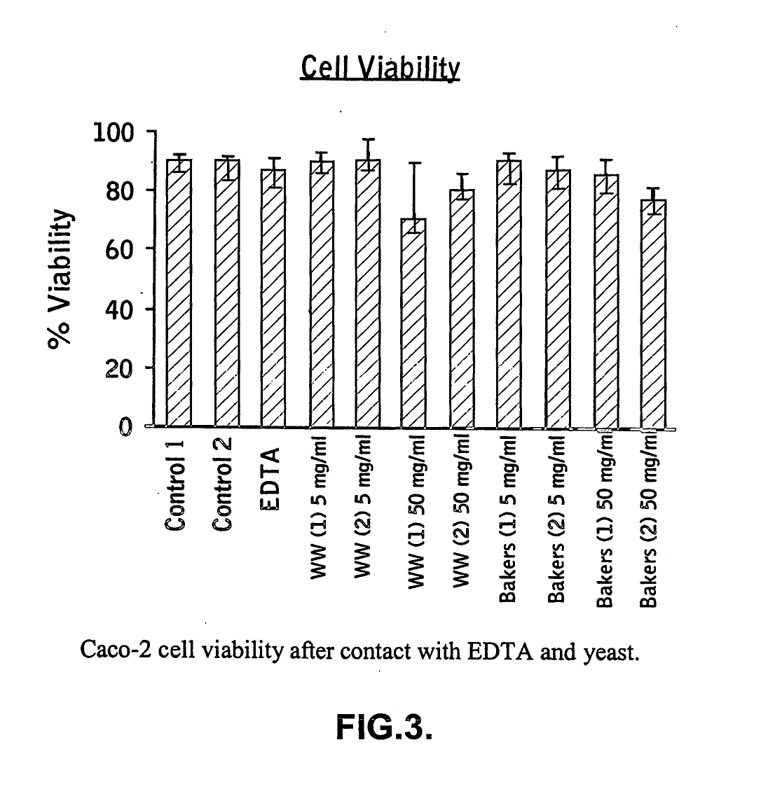

FIG. 3 shows the viability of Caco-2 cells after-incubation with EDTA and yeast in the different concentrations.

### EXAMPLE 1

Caco-2 cells (passage 47-50 derived from a human colon carcinoma cell line, which serve as an in vitro model for the intestinal epithelium) were grown in Dulbeccos modified eagle medium supplemented with 10% foetal calf serum, 0.5 μm/ml penicillin and 0.1 mg/ml streptomycin. Once the cells had reached 80% confluence the growth medium was removed, the cells washed with phosphate buffer solution (pH 7.4) and separated from the surface and each other by the addition of Trypsin-EDTA solution. Growth medium was then added to the flask to inhibit the Trypsin enzymatic reaction and the cells were counted using a haemocytometer and suspended in fresh growth medium. Previously calculated amounts of cell suspensions were added to each cell insert and fresh Dulbecco's medium added to both the apical and basolateral layer. Once the cells became 100% confluent (approx. 10 days), spray-dried yeast suspended in growth medium was added to the apical side of each insert. The trans-epithelial electrical resistance (TEER), a measure for the tightness of the cell layer, was subsequently measured at specific time intervals. Once the TEER experiments were completed the growth medium was removed and the Caco-2 cells washed with phosphate buffer solution and tested for viability using tryptan blue solution (all reagents etc. were obtained from Sigma-Aldrich, Fancy Road, Poole, Dorset, BH12 4QH, UK).

FIG. 1 demonstrates the effect of Washed Williams yeast (5 and 50 mg/ml, spray-dried product) on cell tight junctions. From this plot an 80% drop in resistance for the higher concentration is shown which in real terms means the TEER is about 20-25% of its original value at complete cell confluence.

### EXAMPLE 2

Caco-2 cells were prepared, exposed to yeast, TEER measured and their viability determined in accordance with the protocol outlined in Example 1 using Bakers yeast (5 and 50 mg/ml) rather than Washed Williams yeast.

FIG. 2 shows the effect of Bakers yeast (5 and 50 mg/ml, spray-dried product) on the tight junctions of Caco-2 cells. Again, a drop in the TEER implies that this yeast type also has an effect on the cell monolayer. However, this effect is different to the effect produced by Williams yeast in that the TEER only drops to about 40% of its original value at the higher concentration and to 65% at the lower concentration. Possible reasons for the difference in yeast types are for example, the different morphologies of the different yeast strains, different concentrations of chitin on the yeast surface, the way in which the different yeast strains have been processed etc.

### EXAMPLE 3

Caco-2 cells were prepared as described in Example 1 and incubated with EDTA and yeast in different concentrations.

FIG. 3 shows that the yeast does not compromise the viability of the cells.

The Examples clearly show two important aspects of the present invention. First, a reduction in the TEER suggests that Williams yeast and Balkers yeast opens cell tight junctions of a Caco-2 cell monolayer. This of course can be a precursor to the absorption of certain drugs across the epithelial cells of the gastrointestinal tract into the systemic circulation. Secondly, the effect is dosage dependent—increasing the dose by a factor of ten lowers the TEER from 40% of the original value to about 20%, quite an important effect bearing in mind we have shown EDTA, a calcium chelator, to lower the TEER to about 45% of the original value.

These results clearly demonstrate that the use of an adjuvant as described hereinabove can be safely used for pharmaceutical applications. Both Bakers yeast and Williams yeast divide by budding. When such cells bud, a small cell emerges from the surface, cell wall, of the parent cell. When the bud separates from the parent cell it leaves behind a bud ‘scar’ on the cell wall of the parent cell. This bud scar is rich in chitin and/or chitosan. Typically, budding in Bakers yeast is inhibited after approximately 5 or 6 divisions, leaving 5 or 6 bud ‘scars’. In contrast, Williams yeast is allowed to divide many more times whereby individual cells often have more than 15 bud ‘scars’. It is believed that the chitin and/or chitosan found in the cell wall of fungi, together with other polysaccharides, such as α- and β-glucans, increase the permeability of the tight junctions whilst mitigating the effects of chitin and/or chitosan on the viability of cells.

### REFERENCES

(1) Diamond, J. M. Physiologist 20 (1977), 10-18
(2) Ballard, S. T. et al., Annu. Rev. Nutr. 15 (1995), 35-55
(3) Dragsten, P. R. et al. Nature 294 (1981), 718-722
(4) van Meer, G. And Simons, K. EMBO J. 5 (1986), 1455-1464
(5) Anderson, J. M. and Van Itallie, C. M. Am. J. Physiol. 269 (1995), G467-G475
(6) Sutton, S. C. et al., Pharm. Res. 10 (1993), 1516-1520
(7) MacFadyen, R. J. et al., Clin. Pharmacokinetics 25 (1993), 274-282
(8) Welling, P. G. et al., Biopharm. Drug Dispos. 6 (1985), 147-157
(9) LeCluyse, E. L. et al. J. Pharmacol. Exp. Ther. 265 (1993), 955-962
(10) Borchard, G. et al., Journal of Controlled Release 39 (1996) 131-138
(11) Amidon, G. L., Pharm. Res. 12: 413-420 (1995)

1. A composition comprising:
a pharmaceutically active compound and a non-encapsulating adjuvant, wherein the adjuvant comprises a fungal cell or a fragment thereof.

2. A composition as claimed in claim 1, wherein the fungal cell fragment comprises a fungal cell wall or a part thereof.

3. A composition as claimed in claims 1, wherein the pharmaceutically active compound comprises a hydrophilic moiety.

4. A composition as claimed in claim 3 wherein the pharmaceutically active compound is hydrophilic or substantially hydrophilic.

5. A composition as claimed in claim 3, wherein the pharmaceutically active compound comprises a peptide.

6. A composition as claimed in claim 5 further comprising proteinase inhibitor.

7. A composition as claimed in claim 1, wherein the fungal cell or fungal cell fragment is derived from one or more fungi selected from the group consisting of Mastigomycotina, Zygomycotina, Ascomycotina, Basidiomycotina and Dezitero7izycotina.

8. A composition as claimed in claim 7 wherein the fungal cell or fungal cell fragment is derived from Ascomycotina.

9. A composition as claimed in claim 8 wherein the fungal cell or fungal cell fragment is derived from yeast.

10. A composition as claimed in claim 9 wherein the fungal cell or fungal cell fragment is derived from one or more selected from the group consisting of Candida albicans, Blastomyces dermatitidis, Coccidioides immitis, Hispltoplasma capsulatum, Paracoccidioides brasiliensis, Penicillium marneffei and Saccharomyces cerevisiae.

11. A composition as claimed in claim 10 wherein the fungal cell or fungal cell fragment is derived from Saccharomyces cerevisiae.

12. A composition as claimed in claim 10, wherein the fungal cell or fungal cell fragment contains chitin and/or chitosan.

13. A composition as claimed in claim 12, wherein the fungal cell or fungal cell fragment contains chitin in an amount of at least about 5% by dry weight.

14. A composition as claimed in claim 12, wherein the fungal cell or fungal cell fragment contains chitosan in an amount of at least about 5% by dry weight.

15. A composition as claimed in claim 12, wherein the pharmaceutically active compound is absorbed via the paracellular pathway.

16. A composition as claimed in claim 12, wherein the adjuvant is present in an amount effective to increase the permeability of the paracellular pathway in vivo.

17. A pharmaceutical composition comprising a composition as claimed in claim 1 and a pharmaceutically active compound encapsulated within a yeast cell.

18. A composition as claimed in claim 1 for use as a medicament.

19. A packaged composition comprising:
at least two discrete components, the first component comprising a pharmaceutically active composition and the second component comprising a fungal cell or a fragment thereof.

20. A method of administering a pharmaceutically active compound via the paracellular pathway comprising the use of a composition as claimed in claim 1.

21. The use of a non-encapsulating fungal cell or fungal cell fragment to augment the bioavailability of a pharmaceutically active compound.

22. The use of a composition as claimed in claim 1 for alleviating the symptoms of and/or curing an ailment or disease.
